(19) United States
(12) Patent Application Publication
(10) Pub. No.: US 2008/0195095 A1
(43) Pub. Date: Aug. 14, 2008

(54) Resilient External Fixator Between Two Bone Portions

(76) Inventors: Xavier Renard, Vauhallan (FR); Philippe Pelissier, Merignac (FR)

Correspondence Address: YOUNG & THOMPSON, 209 Madison Street, Suite 500, ALEXANDRIA, VA, 22314, US

(21) Appl. No.: 11/914,455

(22) PCT Filed: May 15, 2006
(86) PCT No.: PCT/FR06/01084

§ 371 Date: Nov. 15, 2007

(30) Foreign Application Priority Data
May 26, 2005 (FR) 0505289

(51) Int. Cl. A61B 17/00

(52) U.S. Cl. 606/54

(57) ABSTRACT

The present invention relates to resilient external fixators between first and second bone portions O₁, O₂. The fixator of the invention is essentially characterized by the fact that it comprises a helical spring (11) having an axis (12), a body (14), first means (15) for mounting the body (14) to co-operate with the spring (11) so that the spring is suitable for turning relative to the body about the axis (12), a pin (16) of axis (17) suitable for being fastened on the bone O₁, means (19) for mounting the pin (16) to co-operate with the body (14) so as to pass through the spring (11), and so that the axis (17) forms a non-zero angle with the axis (12), another body (22), and means (24) for connecting the body (22) with the bone O₂ and the spring (11). The invention is applicable specifically to external fixators for fingers.

The present invention relates to resilient external fixators suitable for being mounted to co-operate with two bone portions between which it is desired to exert traction or else distraction, and finding a particularly advantageous application in treating joint fractures by distraction on the principle of ligamentotaxis in which the traction exerted on either side of the fracture reduces the displacement of the fragments and holds them in a position suitable for encouraging remodeling of the joint.

Such external fixators already exist, and they find applications in particular in the field of repairing finger bones. Such a fixator essentially comprises two hooks interconnected by a resilient spring and suitable for being fastened respectively to the two bone portions. The hooks are of two types: they are rectilinear in shape if it is desired to exert traction, and substantially Z-shaped if it is desired to exert distraction by means of an elastic traction force.

Presently-known external fixators do not enable the magnitude of the force exerted between the two bone portions to be modulated, whether in traction or in distraction, and they require at least two different embodiments depending on the type of force it is desired to exert.

Those known systems are essentially hand-built assemblies that are difficult to make and that depend greatly on the operator. The results of that known technique are therefore unpredictable. Furthermore, the assemblies that are made are generally bulky. The same applies to external fixators that are converted away from their simple fastening function and made to be mobile. Their volume is associated with the fact that the systems for fastening to the bone, the systems exerting distraction, and the systems maintaining the axis, e.g. of the finger, are all independent, with each system occupying its own volume.

Thus, an object of the present invention is to provide a resilient external fixator having a single structure regardless of the type of force that is to be exerted between the two bone portions, i.e. traction or distraction, and that enables the magnitude of the force to be modulated, making joint mobilization possible at the same time as distraction.

More precisely, the present invention provides a resilient external fixator between first and second bone portions, the fixator being characterized by the fact that it comprises:

a helical spring defined along a first axis;

a first body;

first means for mounting said first body in co-operation with said helical spring so that said helical spring is suitable for turning relative to said first body about the first axis;

a first pin suitable for being fastened to said first bone portion, said first pin being oblong in shape and defined along a second axis;

means for mounting said first pin to co-operate with said first body so that it passes through said helical spring and so that said second axis forms a non-zero angle with the first axis;

a second body; and

means for connecting said second body with said first bone portion and said helical spring.

Other characteristics and advantages of the invention appear from the following description given with reference to the accompanying drawings by way of non-limiting illustration, and in which:

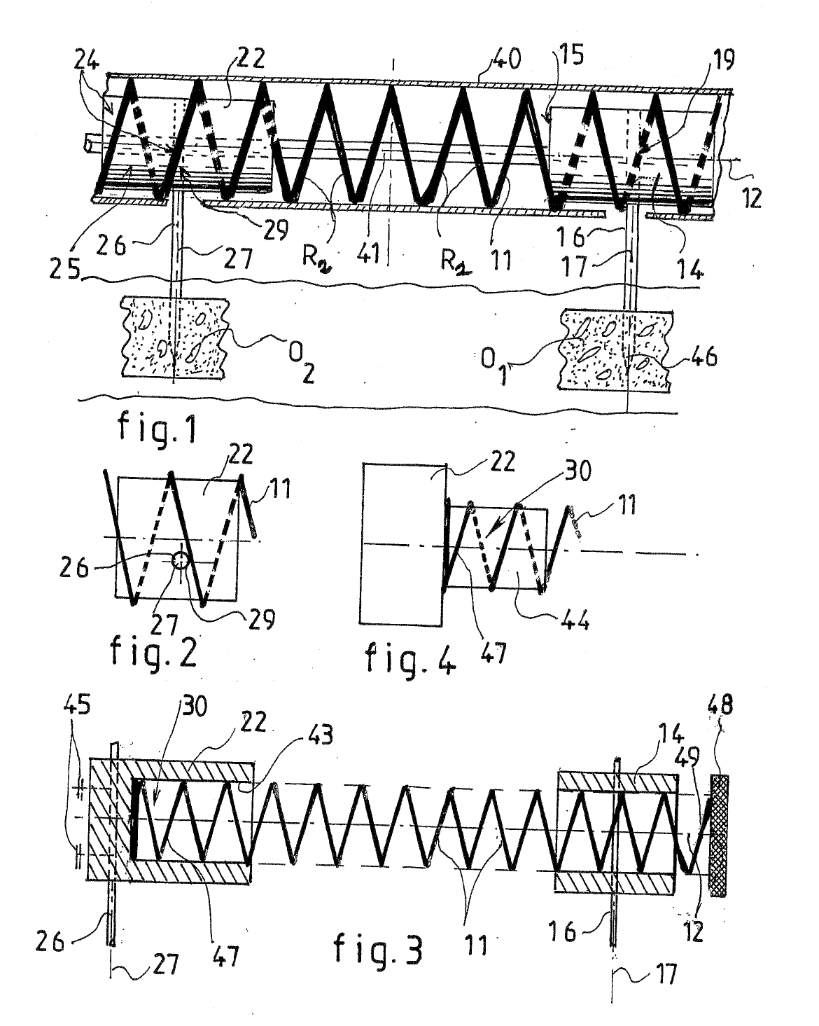

FIGS. 1 and 2 are two orthogonal diagrammatic views of a first embodiment of the external fixator of the invention;

FIG. 3 is a diagrammatic view of a second embodiment of the external fixator of the invention;

FIG. 4 is a diagrammatic view of another embodiment of a portion of the external fixator in the embodiment shown in FIG. 3, i.e. the portion associated with mounting one end of the helical spring in co-operation with a body;

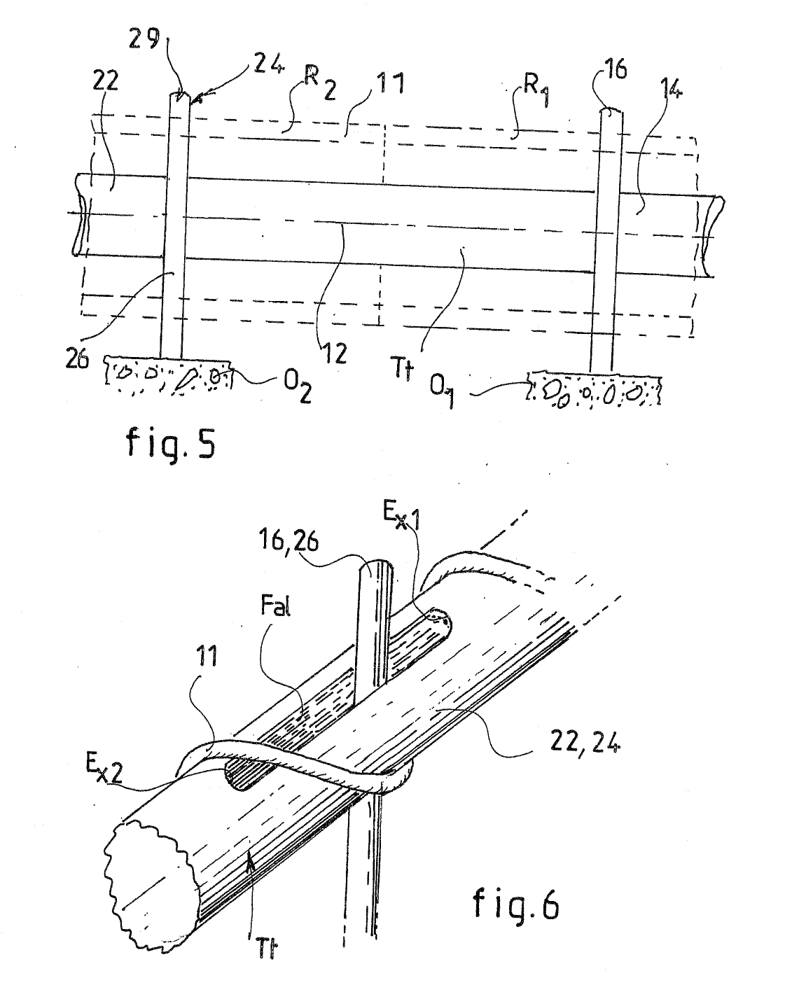

FIGS. 5 and 6 are diagrams showing two other improved embodiments of an external fixator of the invention.

It is stated initially that, in the figures, the same references are used to designate the same elements, regardless of the figure in which the elements appear and regardless of the way in which the elements are shown. Similarly, if elements are not specifically referenced in one of the figures, their references can easily be discovered by referring to another figure.

It is also stated that the figures show essentially four embodiments of the subject matter of the invention, but that other embodiments can also exist that comply with the definition of the invention.

With reference to FIGS. 1 to 6, the invention described below relates to an external fixator suitable for exerting a resilient force of adjustable intensity between first and second bone portions O₁ and O₂, and it finds a particularly advantageous application as an external fixator for exerting traction or distraction, both of which can be modulated, between two bones or two fractions of a finger bone, e.g. between two phalanges.

In all of the embodiments shown in FIGS. 1 to 4, the external fixator essentially comprises, in its primary definition: a helical spring 11 defined along a first axis 12; a first body 14; first means 15 for mounting said first body in co-operation with the helical spring 11 so that the helical spring is capable of pivoting relative to the first body substantially about the first axis 12; a first pin 16 suitable for being fastened to the first bone portion O₁, said first pin 16 being oblong in shape along a second axis 17; and means 19 for mounting the first pin 16 to co-operate with the first body 14 so that it passes through the helical spring 11 and so that the second axis 17 forms a non-zero angle with the first axis 12.

In an advantageous embodiment, the pin 16 is a bone pin that is well known in itself, of circularly cylindrical or similar shape, and having an end 46 for penetrating into a bone, which end includes a bone thread, e.g. of the self-tapping type.

By way of example, the means 19 for mounting the first pin 16 to co-operate with the first body 14 are constituted by a through orifice made in the body 14 and of cross-section complementary to that of the pin 16, and advantageously by means for locking the pin in the through orifice and thus relative to the first body 14, such as a fastener screw or the like, represented diagrammatically by way of illustration at 45 in FIG. 3 in the application to fastening the pin 26 on the second body 22 in the manner described below.

The external fixator of the invention also includes a second body 22 and means 24 for connecting the second body 22 with the second bone portion O₂ and with the helical spring 11.

In the embodiment shown in FIGS. 1 and 2, the means 24 for connecting the second body 22 with the second bone portion O₂ and the helical spring 11 comprise: second means 25 for mounting the second body 22 to co-operate with the helical spring 11 so as to allow the spring to pivot relative to the second body about the first axis 12; a second pin 26 suitable for being fastened to the second bone portion O₂ and defined along a third axis 27; and means 29 for mounting the second pin 26 to co-operate with the second body 22 so as to pass through the helical spring 11 and so that the third axis 27 forms a non-zero angle relative to the first axis 12.

FIGS. 1 and 2 show a first embodiment of the external fixator of the invention, in which the two bodies 14 and 22 are substantially identical and advantageously circularly cylindrical, and the two pins 16 and 26 pass through the helical spring 11 and through respective ones of the two bodies 14 and 22, co-operating with those two bodies and the helical spring in the same manner.

In an advantageous embodiment, the helical spring 11 has non-touching turns and it is made of a material such as stainless steel, or the like.

The first and second above-defined means 15 and 25 for mounting the bodies 14 and 22 respectively in co-operation with the helical spring 11 so as to allow it to pivot relative to the bodies about the first axis 12 are constituted by the fact that the helical spring 11 is mounted on the bodies in one of the following two positions: a first position in which it surrounds the body, as shown in FIG. 1, or a second position in which it passes through the body along an axial bore, as shown in FIG. 3.

It is specified that according to this characteristic of the invention, the helical spring may be mounted relative to both bodies 14 and 22 in the same position, e.g. by surrounding both of them as shown in FIG. 1 for mounting on the two bodies 14 and 22, or else on one of the two bodies in one of the two positions, e.g. by passing through it on an axial bore as shown in FIG. 3 for mounting to the first body 14, while being mounted to the other body in the other position, i.e. in the above example, by surrounding it, as shown in FIG. 1 for mounting on the second body 22.

In the embodiment shown in FIG. 1, the helical spring 11 with non-touching turns, as defined above, has two consecutive portions R₁ and R₂ with the turns in one of the two portions being wound in the opposite direction to the direction in which the turns are wound in the other portion.

With reference to FIG. 2, it can be seen that the turns of the spring 11 are equivalent to a female thread of variable pitch, while the pin, e.g. 26, is equivalent to a point on a complementary male thread that is suitable for following the “ramp” constituted by the female thread. In this way, by causing the spring 11 to pivot about its axis 12, the spring is caused to move in rotation and in translation relative to the pin 26, assuming that the pin is stationary. With reference to the embodiment of FIG. 1, since the two spring portions R₁ and R₂ are wound in opposite directions, the two pins 16, 26 will either be moved towards each other or apart from each other, depending on the direction in which the spring 11 is turned. However if the two pins are considered as being stationary, the number of turns of the spring that extend between them will either increase or decreased, thereby varying the tension force exerted by said portion of the spring on the two pins.

The embodiment described above and shown in FIGS. 1 and 2 thus enables the traction or distraction force that is exerted on the two bone portions O₁ and O₂ to be adjusted depending on the direction in which the helical spring 11 is turned about its axis 12 relative to the two bodies 14 and 22, as explained below.

FIG. 3 shows another embodiment of the external fixator of the invention.

In this other embodiment, the non-touching turns of the helical spring 11 are all wound in the same direction, and the first and second bodies 14, 22 are mounted around the spring.

As in the first embodiment shown in FIGS. 1 and 2, the fixator comprises a first bone pin 16 and means 19 for mounting said first pin 16 to co-operate with the first body 14, said first pin 16 and said means 19 being like those described above for the first embodiment.

The means 24 for connecting the second body 22 with the second bone portion O₂ and the helical spring 11 comprise means 30 for mounting one end 47 of the helical spring 11 to rotate in a plane relative to the second body 22 and means for fastening said second body to the second bone portion O₂, which means are constituted, as in the first embodiment of the fixator, by a second pin 26 that is secured both to the second body 22 and to the second bone portion O₂.

Two embodiments of the above-defined means 30 are shown respectively in FIGS. 3 and 4.

In the embodiment of FIG. 3, these means 30 are constituted by the fact that the end 47 of the helical spring 11 is mounted in a hollow housing 43 formed in the second body 22. The spring can thus turn relative to the second body, and even more easily when it is turned in the direction for compressing it, i.e. when it is acting in traction on the two bone portions O₁ and O₂, since under such circumstances, its outside diameter decreases.

In the embodiment of FIG. 4, these means 30 are constituted by the fact that the end 47 of the spring 11 is mounted around a projecting portion 44 secured to the second body 22. The spring can thus pivot relative to the second body, even more easily when it is pivoted in the direction for decompressing it, i.e. when it is acting in distraction on the two bone portions O₁ and O₂, since under such circumstances, its outside diameter increases.

The external fixator may also include grip means 48 that are mounted in association, e.g. with one end 49 of the helical spring 11, so as to make it easier to pivot it about its axis 12. By way of example, these grip means can be constituted, as shown in FIG. 3, by a knob 48 or the like, and advantageously one that is knurled or the like.

In order to make the external fixator of the invention easier to produce industrially, and easier to put into place and be operated, it is advantageous for the second and third axes 17, 27 to be mutually parallel and perpendicular to the first axis 12, as shown in all of the figures.

The external fixator may also include a rigid or resilient protective sheath 40 (FIG. 1) surrounding the outside of the helical spring 11, and/or a rigid longitudinal rod 41 passing through at least one of the two bodies 14 and 22, e.g. being fixed to one of them, e.g. the body 14 in FIG. 1, and slidably in a complementary hole formed in the other body 22.

The means described above also serve to hold the spring substantially rectilinear along its axis 12 and to prevent it from being subjected to arcuate deformation when it exerts a force couple, whether in lengthening or in compression, on the two bodies 14 and 22.

The external fixator of the invention operates and is used as follows.

The description of the operation and the use of the external fixator begins with reference to the embodiment shown in FIG. 1.

It is assumed that traction or distraction forces need to be exerted on the two bone portions O₁ and O₂. To do this, the two bodies 14, 22 are slid into the spring 11 and positioned on either side of the change in winding direction of the turns R₁, R₂. The two bodies are brought into register with the two bone portions O₁ and O₂. The two pins 16, 26 are positioned respectively in the two bodies, passing through the spring as described above, with their threaded ends being screwed into the corresponding bone portions.

For example by means of set screws, they are then advantageously secured to their respective bodies. It can also be assumed that one of the bodies 14, 22 (or both of them) is merely mounted to slide on a pin 16, 26, said pin advantageously including an abutment or the like, e.g. at its non-threaded end, so as to prevent the body from escaping from its pin.

The spring is in the rest position and the portion of the spring extending between the two pins 16, 26 has a certain number of turns, specifically seven in FIG. 1.

If it is desired to exert traction between the two bone portions O₁ and O₂, then the spring is turned about its axis 12 so that the number of turns between the two pins decreases.

In this way, the spring portion extending between the two pins stretches and exerts two traction forces in opposite directions that are applied via the pins on the two bone portions. By turning the spring to a greater or lesser extent about its axis 12, it is thus possible to modulate the intensity of the traction exerted between the two bone portions.

If it is desired to exert distraction between the two bone portions O₁ and O₂, then the spring is turned about its axis 12 so as to increase the number of turns between the two pins. In this way, the spring portion extending between the two pins becomes compressed and exerts two opposite distraction forces on the pins that are applied via the pins to the two bone portions. By turning the spring to a greater or lesser extent about its axis 12, it is possible to modulate the intensity of the distraction exerted between the two bone portions.

With the embodiment of FIGS. 3 and 4, it is also possible to exert traction or distraction between the two bone portions. However, if it is desired to exert traction, it is then necessary to mount the bodies 14, 22 and the pins 16, 26 in association respectively with the two bone portions O₁ and O₂ so that the portion of spring that extends between the two pins is initially stretched to a relatively large extent. The fixator then exerts on the two bone portions a traction force that is greater than that desired, but by turning the spring so as to increase the number of turns between the two pins, it is possible to adjust this traction.

If it is desired to exert distraction between the two bone portions O₁ and O₂, it is necessary to mount the two bodies 14, 22 and the pins 16, 26 in association respectively with these two bone portions so that the portion of spring that extends between the two pins is initially compressed to a relatively large extent. The fixator then exerts a distraction force on the two bone portions that is greater than the desired force, but by turning the spring so as to increase the number of turns between the two pins, it is possible to adjust this distraction.

FIGS. 5 and 6 show two other embodiments of the fixator of the invention, which are variants equivalent to those described above with reference to FIGS. 1 to 4.

In particular, FIG. 5 shows a variant of the fixator in which the first body 14 and the second body 22 are made directly in a single rigid or flexible rod Tₜ, and the spring 11 is formed in the same manner as shown in FIG. 1, i.e. with two consecutive portions R₁ and R₂ that are wound in opposite directions.

The means 19, 29 for mounting the first and second pins 16, 26 to co-operate with the rod Tₜ are constituted by the fact that each pin passes between a turn and the rod Tₜ. Naturally, one of the two pins 16 passes through the spring portion R₁ and the other pin 26 passes through the spring portion R₂. In this embodiment, the two pins move along the rod Tₜ, rubbing against its side wall, depending on whether the spring 11 is turned in one direction or the other, as described above.

FIG. 6 shows another variant of the fixator of the invention, which can in fact be an improvement of the variant shown in FIG. 5.

As described above, in the embodiment of FIG. 5, when the spring 11 is turned, the two pins can move relative to the rod Tₜ, without limit. In order to limit the amplitude of the traction or the distraction between the two bone portions O₁, O₂, at least one of the two pins 14, 16 passes through an oblong slot Fₐₗ formed in the rod Tₜ along its longitudinal axis 12, as shown in FIG. 6.

In this embodiment, the amplitude of the traction or the distraction is limited by the fact that the pin 14 or 16 comes into abutment against one or the other of the end walls Eₓ₁, Eₓ₂ of the oblong Fₐₗ.

1. A resilient external fixator between first and second bone portions (O₁, O₂), the fixator being characterized by the fact that it comprises:
a helical spring (11) defined along a first axis (12);
a first body (14);
first means (15) for mounting said first body (14) in co-operation with said helical spring (11) so that said helical spring is suitable for turning relative to said first body about the first axis (12);
a first pin (16) suitable for being fastened to said first bone portion (01), said first pin (16) being oblong in shape and defined along a second axis (17);
means (19) for mounting said first pin (16) to co-operate with said first body (14) so that it passes through said helical spring (11) and that said second axis (17) forms a non-zero angle with the first axis (12);
a second body (22); and
means (24) for connecting said second body (22) with said first bone portion (02) and said helical spring (11).

2. A fixator according to claim 1, characterized by the fact that the means (24) for connecting said second body (22) with said second bone portion (O₂) and said helical spring (11) comprise:
second means (25) for mounting said second body (22) in co-operation with said spring so that said helical spring is suitable for turning relative to said second body about the first axis (12);
a second pin (26) suitable for being fastened to said second bone portion, said second pin being oblong in shape and defined along a third axis (27); and
means (29) for mounting said second pin to co-operate with said second body so as to pass through said helical spring (11) and so that said third axis (27) forms a non-zero angle with the first axis (12).

3. A fixator according to claim 2, characterized by the fact that said helical spring (11) has non-touching turns.

4. A fixator according to claim 3, characterized by the fact that said helical spring (11) comprises two consecutive portions (R₁, R₂), the turns in one of the two portions being wound in the opposite direction to the direction in which the turns in the other portion are wound.

5. A fixator according to claim 2, characterized by the fact that the first and second means (15, 25) for mounting the bodies (14, 22) in co-operation with said helical spring (11) so that said helical spring is suitable for turning relative to said bodies about the first axis (12) are constituted by the fact that said helical spring (11) is mounted on said bodies in one of the following two positions: a first position in which it passes through said body in an axial bore; a second position in which it surrounds said body.

6. A fixator according to claim 1, characterized by the fact that the means (24) for connecting said second body (22) with said second bone portion (O₂) and said helical spring (11) comprise means (30) for mounting one end (47) of said helical spring (11) to turn in a plane relative to said second body (22), and means for fastening said second body with said second bone portion (O₂).

7. A fixator according to claim 1, characterized by the fact that said helical spring acts in one of the following two manners: in traction; in distraction.

8. A fixator according to claim 1, characterized by the fact that it includes grip means mounted in association with said helical spring to cause it to turn about the first axis (12).

9. A fixator according to claim 2, characterized by the fact that the second and third axes (17, 27) respectively of the first and second pins (16, 26) are mutually parallel and perpendicular to the first axis (12).

10. A fixator according to claim 1, characterized by the fact that, by way of application, it constitutes an external fixator between two bone points in a finger of a human being.

11. A fixator according to claim 1, characterized by the fact that it includes means for keeping said spring (11) substantially rectilinear along its axis (12).

12. A fixator according to claim 4, characterized by the fact that both bodies (14, 22) are formed in a single rod (Tₜ).

13. A fixator according to claim 12, characterized by the fact that at least one of the two pins (14, 26) passes through said rod in an oblong slot (Fₐₗ) extending along the longitudinal axis (12) of said rod (Tₜ).

14. A fixator according to claim 4, characterized by the fact that the first and second means (15, 25) for mounting the bodies (14, 22) in co-operation with said helical spring (11) so that said helical spring is suitable for turning relative to said bodies about the first axis (12) are constituted by the fact that said helical spring (11) is mounted on said bodies in one of the following two positions: a first position in which it passes through said body in an axial bore; a second position in which it surrounds said body.

15. A fixator according to claim 14, characterized by the fact that both bodies (14, 22) are formed in a single rod (Tₜ).

16. A fixator according to claim 3, characterized by the fact that the first and second means (15, 25) for mounting the bodies (14, 22) in co-operation with said helical spring (11) so that said helical spring is suitable for turning relative to said bodies about the first axis (12) are constituted by the fact that said helical spring (11) is mounted on said bodies in one of the following two positions: a first position in which it passes through said body in an axial bore; a second position in which it surrounds said body.

17. A fixator according to claim 3, characterized by the fact that the second and third axes (17, 27) respectively of the first and second pins (16, 26) are mutually parallel and perpendicular to the first axis (12).

18. A fixator according to claim 4, characterized by the fact that the second and third axes (17, 27) respectively of the first and second pins (16, 26) are mutually parallel and perpendicular to the first axis (12).

19. A fixator according to claim 2, characterized by the fact that, by way of application, it constitutes an external fixator between two bone points in a finger of a human being.

20. A fixator according to claim 2, characterized by the fact that it includes means for keeping said spring (11) substantially rectilinear along its axis (12).
